(19) United States
(12) Patent Application Publication
(10) Pub. No.: US 2012/0209206 A1
(43) Pub. Date: Aug. 16, 2012

(54) CATHETER-SECURABLE CUFF

(75) Inventor: Charles Scandone, JR., Malvern, PA (US)

(73) Assignee: MEDICAL COMPONENTS, INC., Harleysville, PA (US)

(21) Appl. No.: 13/370,487

(22) Filed: Feb. 10, 2012

Related U.S. Application Data
(60) Provisional application No. 61/441,468, filed on Feb. 10, 2011.

(51) Int. Cl. A61M 25/04

(52) U.S. Cl. 604/175

(57) ABSTRACT

A tissue-ingrowth cuff for stabilization of an implanted catheter against movement, that is placeable onto the catheter by the practitioner at a desired location. The cuff is actuated by the practitioner once moved to its desired location, to grip the catheter. The cuff comprises two body portions securable to each other about a catheter at a practitioner-selected location, by means of complementary locking sections. The two cuff portions are separately slidable along the catheter prior to coupling, but include complementary catheter-gripping sections that cooperate with each other to grip the catheter upon assembly to each other. Tissue-ingrowth material is affixed to the outwardly facing surface of the cuff within a subcutaneous tunnel after catheter implantation and tunneling, thus anchoring the catheter against movement thereafter.

This application claims the benefit of U.S. Provisional Application No. 61,441,468 filed on Feb. 10, 2011, the contents of which are incorporated herein by reference.

### FIELD OF THE INVENTION

This relates to the field of medical devices and more particular to implantable catheters.

### BACKGROUND OF THE INVENTION

Catheters, shunts, drainage tubes and the like, generally referred to herein as catheters, may be located in various locations within the human body for the introduction and removal of fluid from the body. If the catheter is to remain in place for a significant length of time, once in place, such catheters are typically anchored against movement by a stabilizing device or are sutured in place. One common catheter stabilizing device is a tissue-ingrowth cuff, typically made of or covered about its outside surface by a fibrous biocompatible material and which is positioned along the catheter so as to be below the surface of the skin in subcutaneous tissue; the subcutaneous tissue then grows into the fabric of the cuff such that the catheter becomes stabilized in position in the catheterized location.

In some cases, the cuff is positioned on the catheter so that when the distal portion of the catheter is inserted in the body part, such as a blood vessel, the cuff is located in a subcutaneous tunnel that is formed by a tunneling tool, such as a trocar, whether before or after cannulation of the vessel with the distal portion of the catheter. The proximal portion of the catheter having the cuff is drawn through the tunnel by the tool while the tool passes through the subcutaneous tissue, and the cuff is generally sized such that when the procedure is done, the cuff will fit snugly in the tunnel. Such a cuff is disclosed in U.S. Pat. No. 5,830,184. It is generally desired that the cuff be positioned medially along the length of the tunnel.

While providing satisfactory anchoring in some situations, catheters manufactured with fabric cuffs affixed thereto are available only in several set lengths having the cuffs pre-located in areas designed to correspond to average patient sizes. The cuffs thus are not movable along the catheter or adjustable to all body sizes and types for optimal positioning. As most patients vary in size, internal distances involved in subcutaneous placement vary accordingly, deviating from the standard catheter cuff locations commercially available and making stable securement difficult in many cases. In addition, the practitioner may have to adjust the catheter itself within the vein to align the cuff within a stable area for securement. Positioning the catheter is particularly important for proper catheter functioning. For example, if the distal tip of a central venous catheter is not properly located within a vessel, the catheter may cause the condition of cardiac arrhythmia or otherwise inhibit flood flow. If the catheter tip is not properly located within a body cavity there may not be sufficient access to the tip to allow bodily fluid to properly flow through the catheter. Ideally, the precise location of the catheter's distal tip should not depend on the position of the catheter's cuff.

There is a desire by practitioners for the availability of catheters that can be customized to particular patients that have uncommon body sizes or shapes, or have unusual medical conditions. Certain catheters are made available to the practitioners wherein the practitioner may trim the length of the catheter, usually at the proximal end portion thereof since the distal catheter end may have been particularly shaped for optimum performance within the blood vessel or heart atrium. In order to permit tunneling, requisite accessories are added after catheter placement in the patient and after subcutaneous tunneling, such accessories including extension tubes for each lumen of the catheter, or proximal end fittings or luer connectors for each such lumen. Often it is desired to provide a hub at or near the proximal end of the implanted catheter, which hub would protect a joining of extension tubes to the respective catheter lumens and be securable to the patient by suturing to protect strain on the tunneled catheter portion. A hub may also be used at a junction of diverging catheter portions to similarly protect the tunneled catheter portion, and one such attachable hub is disclosed in U.S. Pat. No. 7,261,708, which hub may also be removed and reattached, allowing for catheter assembly repair that does not require removal of the implanted catheter from the patient.

It is desired to provide a tissue ingrowth cuff that is easily attachable to the catheter at an optimal location by the practitioner while at the patient's bedside.

### BRIEF SUMMARY OF THE INVENTION

Briefly, the present invention is a tissue-ingrowth cuff securable to a catheter by the practitioner. The cuff is placed onto and around the catheter and slid along the catheter at a location selected by the practitioner for eventual disposition within a subcutaneous tunnel, and thereafter the cuff is actuated to grip the catheter at that location. Tissue-ingrowth material is preferably pre-affixed to the outwardly-facing surfaces of the cuff body.

In a preferred embodiment, the cuff includes two body portions that are each annular and adapted to be translated along the length of the catheter as desired when separate from each other (or assembled in a first locking condition), until being moved to an optimum location along the catheter such that interface surfaces of each are facing each other. The cuff body portions include complementary locking sections to be coupled to each other, and complementary catheter-gripping sections that cooperate with each other adjacent the catheter to grip the catheter upon the body portions being coupled to each other. At least one locking section is defined on the interface surface of at least one of the body portions, that is lockably engageable with a complementary locking section defined on the interface surface of the other body portion when the two body portions are urged toward and against each other after having been properly oriented to align the locking sections of the body portions. The at least one locking section may be for example a male member or post insertable into a complementary female member or aperture to secure the body portions together, preferably, by a latching projection engageable behind a latching ledge along the aperture. Preferably, there are a pair of such locking section arrangements disposed at opposite sides of the interface surfaces of both body portions, although more than two such arrangements may be utilized.

Further, at least one catheter-gripping section is defined on the interface surface of at least one of the body portions, that is cooperable with a complementary catheter-gripping section of the interface surface of the other body portion when the two body portions are urged toward and against each other. The at least one catheter-gripping section may for example be a male member or projection of one body portion extending toward a complementary female member or opening of the other body portion, whereby the projection urges a flexible wall section of the opening toward and grippingly against the catheter's outer surface when the projection is fully inserted into the opening. Preferably, there are a pair of such catheter-gripping section arrangements disposed at opposite sides of the interface surfaces of both body portions.

A key advantage of the present invention is that a cuff is provided that may be placed onto the catheter by the practitioner at a location therealong selected by the practitioner at the patient's bedside. This advantage enables the practitioner to trim excess length of a deliberately overlong catheter to customize the length to a particular patient's size or condition, in turn obviating the need to have an inventory of catheters of various standard lengths.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is an isometric view of a cuff of a first exemplary embodiment assembled and affixed to a catheter, with tissue-ingrowth material shown partially unwrapped from around the cuff body to reveal details of the cuff body;

FIG. 2 is an isometric view of the cuff of FIG. 1 exploded;

FIGS. 3 to 5 are isometric views of the cuff of FIGS. 1 and 2 being assembled together about a catheter;

FIG. 6 is a plan view of the cuff of FIG. 1 with the cuff body portions positioned to be assembled to each other;

FIG. 7 is a cross-sectional view along the line 7-7 in FIG. 6;

FIG. 8 is similar to FIG. 6 but rotated 90° therefrom about the longitudinal axis;

FIG. 9 is a cross-sectional view along the line 9-9 in FIG. 8;

FIG. 10 is a plan view of the cuff of FIG. 1 with the cuff body portions assembled to each other in a first latching position;

FIG. 11 is a cross-sectional view along the line 11-11 in FIG. 10;

FIG. 12 is similar to FIG. 10 but rotated 90° therefrom about the longitudinal axis;

FIG. 13 is a cross-sectional view along the line 13-13 in FIG. 12;

FIG. 14 is a plan view of the cuff of FIG. 1 with the cuff body portions assembled to each other in a second, final latching position;

FIG. 15 is a cross-sectional view along the line 15-15 in FIG. 14;

FIG. 16 is similar to FIG. 14 but rotated 90° therefrom about the longitudinal axis;

FIG. 17 is a cross-sectional view along the line 17-17 in FIG. 16;

FIG. 18 is a plan view of a catheter assembly including the cuff of FIG. 1, and including extension tubes and a hub;

FIG. 19 is a view similar to FIG. 18 exploded to illustrate the several components of the assembly;

FIGS. 20 and 21 are views similar to FIG. 19, showing the cuff positioned onto the catheter in both the unassembled and assembled states;

FIG. 22 is a view similar to FIG. 21 showing the hub being assembled;

FIGS. 23 and 24 are diagrammatic views of the catheter's distal portion positioned in the blood vessel of a patient and out thereof through a venotomy, with a cuff of FIG. 1 in position on the catheter's proximal portion extending through a subcutaneous tunnel of the patient, and then with the hub and an extension tube assembly being assembled to the catheter proximal end portion extending proximally from the tunnel exit;

FIGS. 25 to 27 are isometric views of a cuff in accordance with an alternative exemplary embodiment in successive assembly positions;

FIG. 28 is a plan view of the cuff of FIG. 25 with the cuff body portions positioned to be assembled to each other;

FIG. 29 is a cross-sectional view along the line 29-29 in FIG. 28;

FIG. 30 is similar to FIG. 28 but rotated 90° therefrom about the longitudinal axis;

FIG. 31 is a cross-sectional view along the line 31-31 in FIG. 30;

FIG. 32 is a plan view of the cuff of FIG. 25 with the cuff body portions assembled to each other in a final latching position;

FIG. 33 is a cross-sectional view along the line 33-33 in FIG. 32;

FIG. 34 is similar to FIG. 32 but rotated 90° therefrom about the longitudinal axis; and

FIG. 35 is a cross-sectional view along the line 35-35 in FIG. 34.

### DETAILED DESCRIPTION OF THE INVENTION

In the drawings, like numerals indicate like elements throughout. Certain terminology is used herein for convenience only and is not to be taken as a limitation on the present invention. The terms “distal” and “proximal” refer, respectively, to directions closer to and away from the patient, and “cuff” refers to a tissue-ingrowth cuff around a catheter for stabilization of an implanted catheter against movement. The terminology includes the words specifically mentioned, derivatives thereof and words of similar import. The terms and expressions used herein, and the embodiments illustrated below, are not intended to be exhaustive or to limit the invention to the precise form disclosed. These terms, expressions and embodiments are chosen and described to best explain the principle of the invention and its application and practical use and to enable others skilled in the art to best utilize the invention.

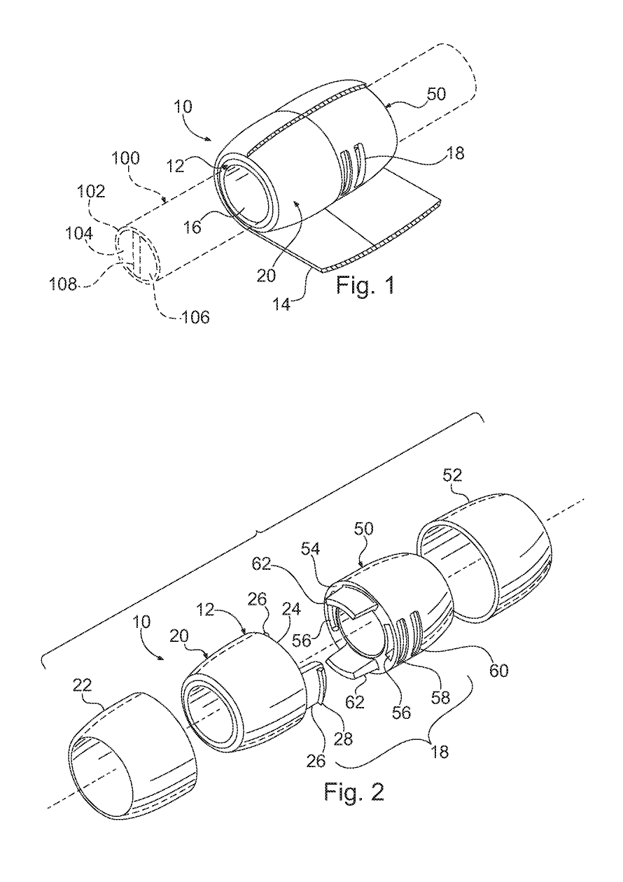

FIG. 1 illustrates a tissue-ingrowth cuff 10, which is an exemplary embodiment of the present invention, in position around a representative catheter 100 that extends through a passageway 16 of the cuff. The catheter 100 is shown as having a side wall 102 and a pair of lumens 104, 106 each having a D-shaped cross-section and separated from each other by a septum 108. Catheter 100 is of the type commonly used for hemodialysis by being implanted into a patient's vasculature; such a catheter is conventionally a flexible tube such as of silicone or polyurethane material, and commonly is manufactured to have a particular type of advantageously shaped and adapted distal end portion. The invention is not limited to use with the illustrated catheter and may be used catheters having various designs. Cuff 10 has a body section 12 and an outer covering 14 of tissue-ingrowth material bonded thereto such as of fibrous DACRON® polyester, with the covering shown partially unwrapped for illustrative purposes only, exposing a latching arrangement 18. The cuff body section 12 may be of a biocompatible material such as polyethylene or polypropylene.

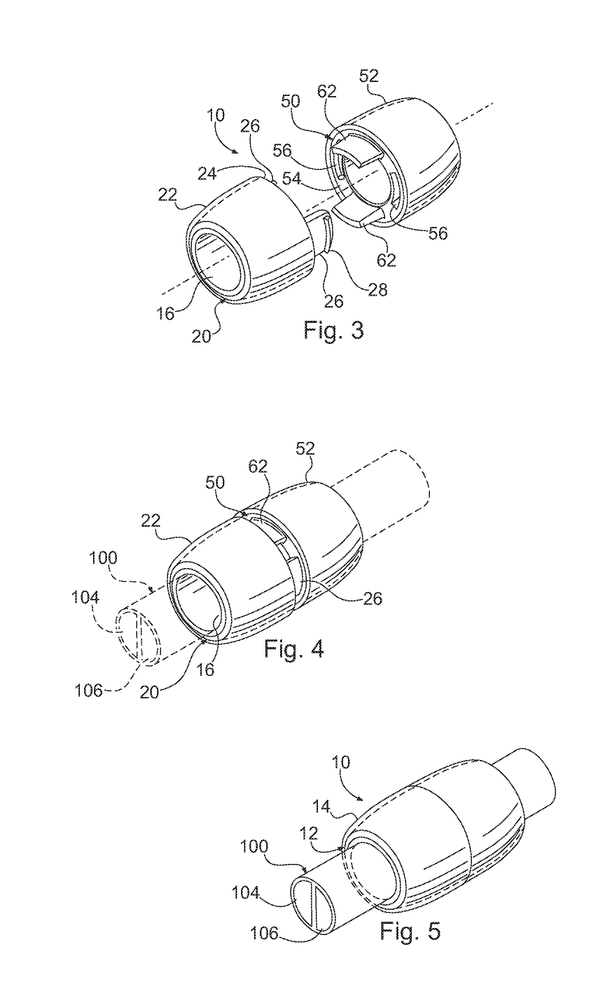

FIGS. 2 to 5 exhibit the exemplary embodiment of cuff 10 wherein the cuff body section comprises a first portion 20 having a first outer covering portion 22 and a first mating interface 24, and a second portion 50 having a second outer covering portion 52 and a second mating interface 54. First and second portions 20, 50 are securable to each other at first and second interfaces 24, 54 by latching arrangement 18, which in the present embodiment provides a first locking position and a second locking position. When cuff 10 is in the first locking position (FIG. 4), the cuff 10 does not grip the catheter 100 but remains slidable therealong; when the cuff 10 is in the second locking position (FIG. 5), the cuff 10 grips the catheter 100, thus preventing relative movement therebetween.

First portion 20 includes at least one, and preferably two opposed, latch arms 26 projecting axially from first interface 24 and having latching projections 28 at free ends thereof. Second portion 50 includes second interface 54 having a latch-receiving aperture 56 for each latch arm 26 which communicates with a pair of axially spaced first position and second position latch windows 58, 60 into each of which the latching projection 28 of the respective latch arm becomes latchable, in sequence as will be explained hereinbelow.

Cuff 10 also includes a catheter-gripping arrangement defined adjacent first and second interfaces 24, 54. Second portion 50 includes at least one, and preferably two opposed, camming arms 62 projecting axially from second interface 54, while first portion 20 includes a camming arm-receiving aperture 32 (see FIG. 13) for receipt of a respective camming arm 62. It is preferable that passageway 16 of the cuff be so dimensioned with respect to the catheter's outer diameter, that a slight amount of friction tends to inhibit slightly the ability of the cuff to slide freely along the catheter.

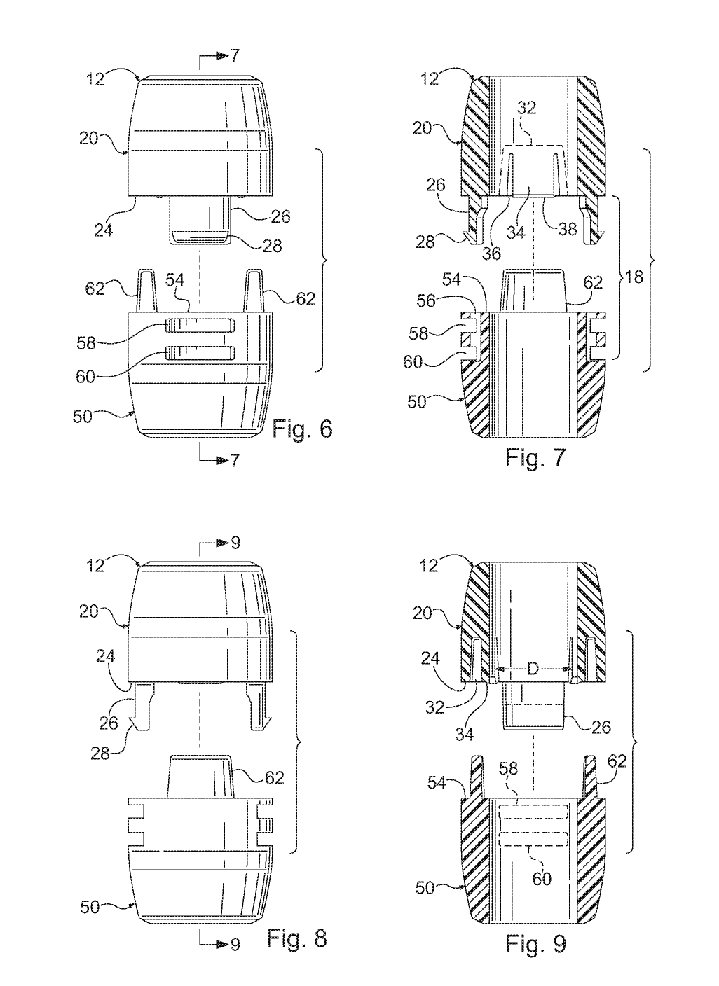

With reference now to FIGS. 6 to 9, the relationship of the latching and catheter-gripping structures of first and second portions 20, 50 may be discerned in greater detail. In FIG. 7, each camming arm-receiving aperture has an inner wall portion 34 adjacent to passageway 16 that is deflectable radially inwardly as a camming arm 62 is received into an aperture 32. Inner wall portion 34 is separated on both sides from the remainder of second portion 50 by gaps 36 enabling such deflection. Free ends 38 of inner wall portions 34 are initially at a natural, non-urged position with a distance D therebetween. As explained hereinafter, upon interconnection of the first and second portions 20, 50, the free ends 38 are fully deflected by camming arms 62 and thereby urged against and into adjacent portions of catheter sidewall 102 to establishing a gripping of the catheter by the cuff. For such camming action, the inner surfaces of camming arms 62 are at an angle from axial that is greater than the angle of the outer surfaces of inner wall portions 34, thus interfering when first and second cuff portions 20, 50 are urged together.

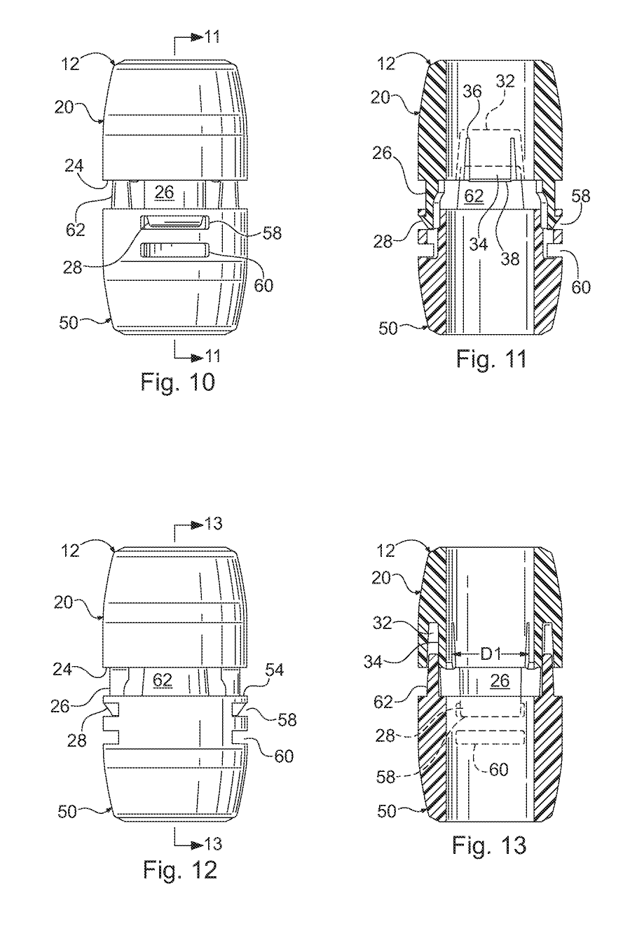

Referring now to FIGS. 10 to 13, body 12 of cuff 10 is shown in the first locking position, where latching projections 28 are latched into corresponding first position windows 58, whereby first and second interfaces 24,54 are spaced apart by a gap. Importantly, in the first locking position, camming arms 62 are only partially inserted into camming arm-receiving apertures 32, with the result that inner wall portions 34 are not yet deflected radially inwardly sufficiently for free ends 38 to grip the catheter sidewall, but preferably the free ends 38 are spaced a distance D1 such that they engage the catheter sidewall 102 to frictionally engage therewith, tending to maintain the cuff 10 in position unless intentionally moved by the practitioner to more precisely position the cuff. The distance D1 may be equal to or smaller than the initial distance D.

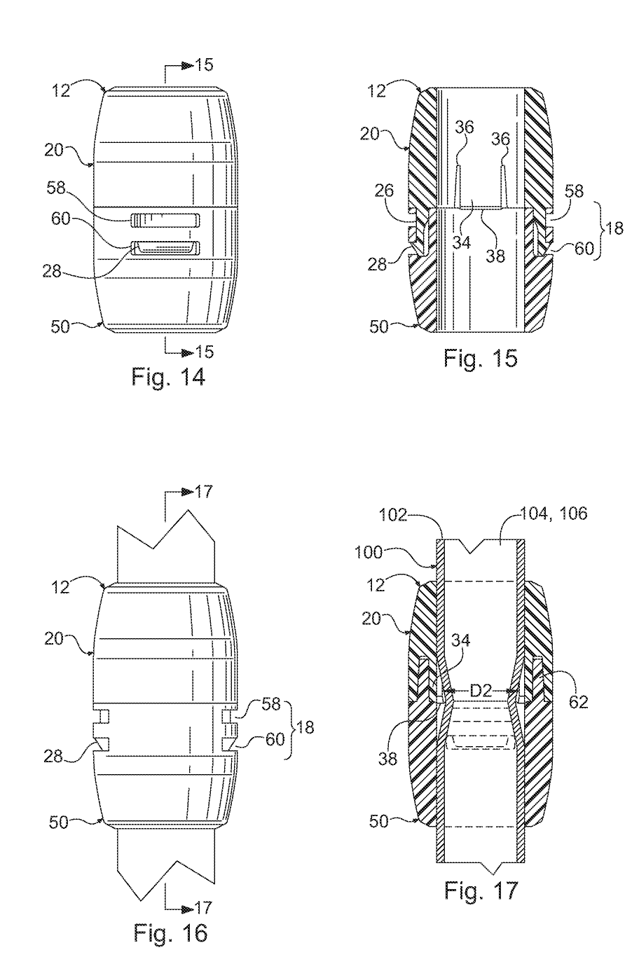

FIGS. 14 to 17 show cuff 10 in the second locking position, wherein the latching projections 28 are latched into second windows 60, first and second interfaces 24,54 are adjacent each other, and camming arms 62 are fully inserted into camming arm-receiving apertures 32. Free ends 38 of inner wall portions are deflected to a distance D2 into gripping engagement with sidewall 102 of catheter 100, as seen in FIG. 17. Distance D2 is smaller than distances D1 and D.

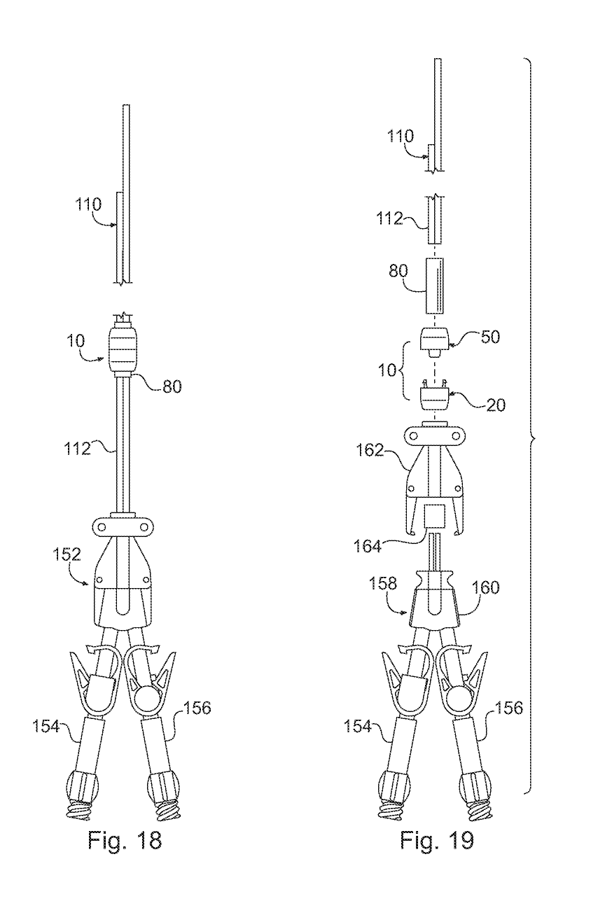

As shown in FIGS. 18-24, to further the gripping engagement when the free ends 38 are at D2, a friction sleeve 80 may be provided about the catheter 100 at approximately the position selected by the practitioner as the cuff site when the catheter is eventually positioned in a subcutaneous tunnel. The sleeve 80 preferably has a length greater than the cuff 10 to provide a range for the final position. The sleeve 80 is preferably manufactured from rubber or some other material providing a higher coefficient of friction than the material of the catheter 100. The sleeve 80 may be formed in a tubular fashion and slid along the catheter or as a flat structure wrapped around the catheter. The sleeve 80 may be maintained in position by frictional forces or may otherwise be secured, for example, by an adhesive applied to the inner surface thereof.

Preferably, cuff portion 20 is more proximal along the catheter than second cuff portion 50. It is seen in FIG. 17 that the edges of free ends 38 of inner walls 34 are almost transverse in orientation. Thus, when the cuff is so arranged, when strain is later placed onto the proximal end portion of an implanted catheter 100 (i.e., pulling on the catheter in a direction away from the patient), the free end edges “bite into” the catheter wall 102 or the sleeve 80 if utilized, thus gripping it more firmly against movement, because the material is pressed against the transverse inner wall free ends 38, while there tends to be less effective gripping of the catheter if the catheter is strained in the opposite direction (i.e., toward the patient), the catheter wall or sleeve encounters only the gently sloping inwardly facing surface of the inner walls 34. Also, preferably, cuff 10 is at an angular orientation around the catheter such that the inner walls 34 are adjacent the ends of the catheter's septum 108 that separates first and second lumens 104, 106, which resists and thereby enhances the cuff's gripping engagement and minimizes impingement of the fluid flow cross-sectional areas of the lumens when gripped by the cuff 10.

FIG. 18 illustrates a complete catheter assembly 150, identifying the catheter's distal end portion 110 and proximal end portion 112, and having a hub 152 by which extension tube subassemblies 154, 156 are joined to the end of catheter proximal end portion 112 for the extension tubes to establish fluid communication with respective ones of first and second lumens 104, 106. FIGS. 19 to 22 illustrate the assembling of catheter assembly 150. In FIG. 19, the various components are shown separately, generally as they would be received by the practitioner at the patient's bedside, including catheter 100 having distal and proximal end portions 110, 112; cuff 10 having first and second portions 20, 50; a friction sleeve 80; an extension tube assembly 158 that includes extension tube subassemblies 154, 156 affixed to a proximal hub portion 160; a distal hub portion 162; and a locking member 164 for locking together the catheter proximal end, the extension tube assembly 158 and distal hub portion 162. The distal and proximal hub portions and their use in assembling a catheter may be as disclosed in U.S. Pat. No. 6,969,381.

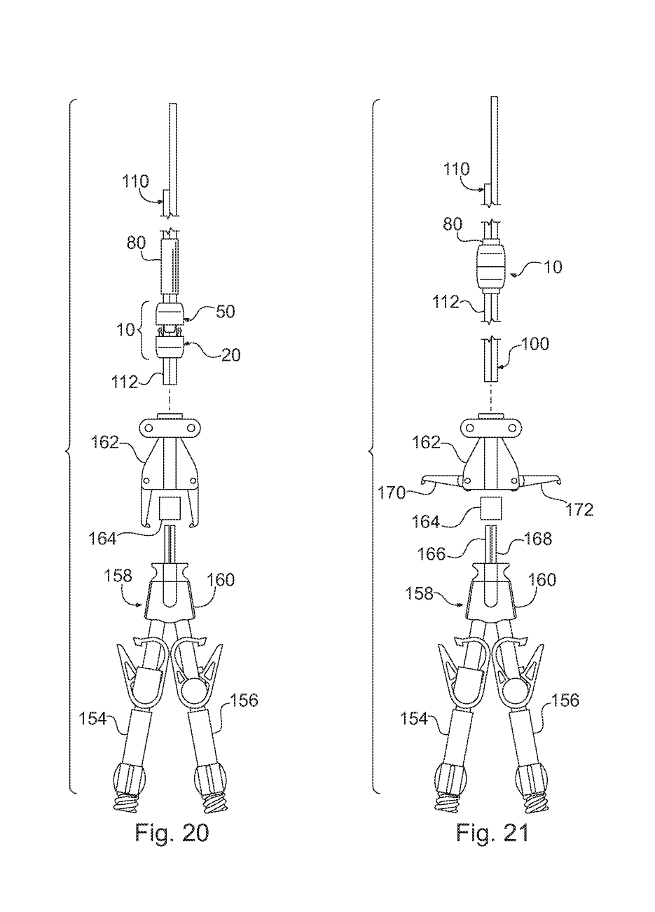

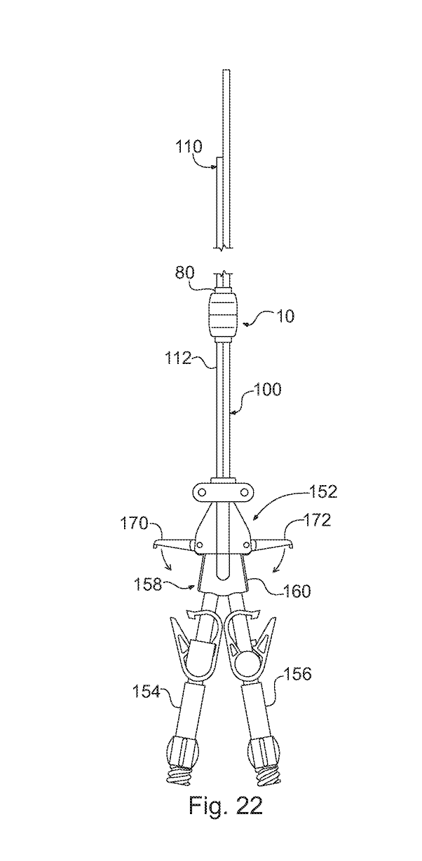

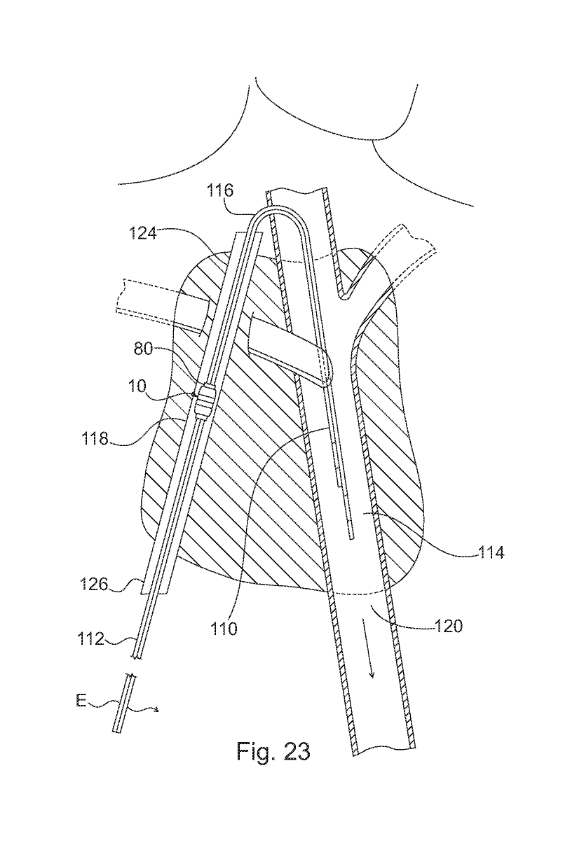

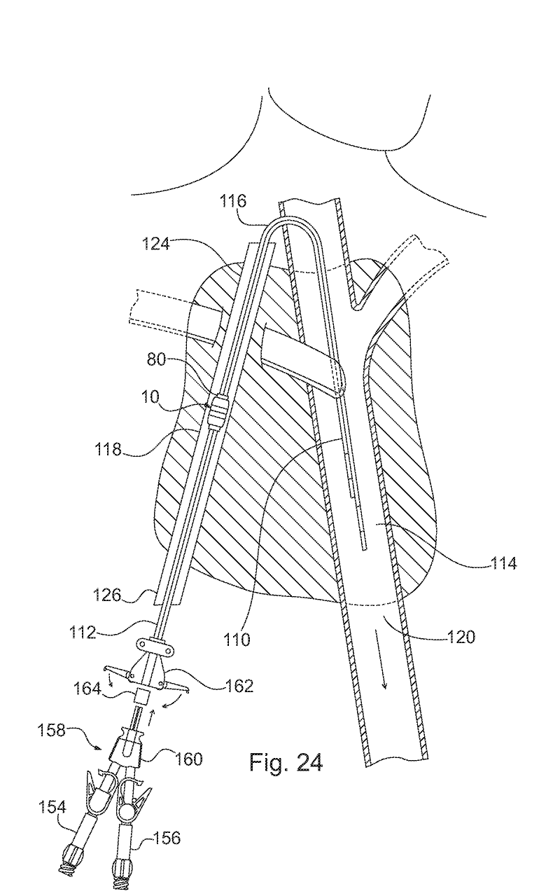

FIG. 20 illustrates that the cuff's first and second portions 20, 50 are then slid onto the end of catheter proximal end portion 112. It should be noted that the cuff portions may be separately placed onto the catheter, or they may first be pre-assembled to each other in their first locking position in which the cuff unit remains slidable along the catheter. As shown in FIGS. 20 and 21, if the sleeve 80 is utilized, it is positioned on the catheter at approximately the position selected by the practitioner for the cuff site when the catheter is eventually positioned in a subcutaneous tunnel (see FIGS. 23 and 24) and then the cuff 10 is slid over the sleeve 80. If the sleeve is not utilized, the cuff 10 is simply slid to the cuff site selected by the practioner. The practitioner trims off the excessive length of the catheter's proximal end portion. Then, as seen in FIG. 22, the distal hub portion 162 and locking member 164 are slid onto the end of the proximal end portion 112, the extension tube assembly 158 with proximal hub portion 160 is brought into position such that cannulae 166, 168 are inserted into ends of the respective catheter lumens 104, 106 and proximal hub portion 160 is urged firmly against distal hub portion 162; cannulae 166, 168 are in fluid communication with respective extension tubes and complete the fluid communication between each extension tube and a respective catheter lumen 104, 106. Finally, the latching arms 170, 172 of distal hub portion 162 are pivoted to lock together the distal and proximal hub portions and also cam distally the proximal hub portion, all as explained in U.S. Pat. No. 6,969,381.

The assembly of the catheter at the patient's bedside is also shown by way of FIGS. 23 and 24 which very generally illustrate implantation of the catheter into the patient. Catheter 100 is adapted to be trimmed by the practitioner at catheter proximal end portion 112, which requires that nothing be attached to the catheter when received by the practitioner at the patient's bedside (except perhaps the cuff in its first locked position located intermediate the distal and proximal catheter end portions 110, 112). Prior to implantation and tunneling and catheter assembly, the practitioner firsts identifies the desired location 114 of the distal tip of the catheter in the vasculature, usually in a major vein 120 adjacent the heart, and also identifies the location of the incision into the skin and into the vein 116 (termed the venotomy). The practitioner identifies the location and length of the eventual subcutaneous tunnel 124 through which the catheter proximal end portion 112 will be disposed for anchoring it in place after implantation.

Then, the practitioner selects the optimum location 118 for the cuff, which will eventually be within the length of the eventual subcutaneous tunnel 124. Once the cuff location 118 is identified along the catheter, preferably the cuff is placed onto the catheter proximal end and finally moved to that identified location 118 while the cuff is in its first locked condition. As explained above, if the sleeve 80 is utilized, it is positioned on the catheter 100 prior to sliding of the cuff 10. When the cuff is positioned in its desired location 118, it is now actuated or locked into its second locked condition and gripping the catheter against further relative movement. Now, the catheter distal end portion may be inserted through the incision and venotomy 116 into the vein, using well-known introducing procedures usually with the assistance of a guide wire (not shown), until the catheter's distal end portion 110 and the distal tip thereof is precisely positioned at desired location 114 within the vasculature. The catheter proximal end portion 112 is then pulled through a subcutaneous tunnel using a tunneler (not shown) according to well-known retrograde tunneling procedures, resulting in the cuff being appropriately positioned within the tunnel. (The practitioner may also choose to tunnel the catheter in an antegrade procedure in which the catheter's distal end portion is pulled through the subcutaneous tunnel prior to being inserted into the vasculature.) The proximal end of the catheter proximal end portion 112 now extends beyond the tunnel exit 126 and the remaining catheter accessories may be assembled onto the catheter 100 to complete the catheter assembly. Excess catheter length E is trimmed and removed by the practitioner prior to placement of the extension tube assembly and hub onto the catheter's eventual proximal end.

Regarding FIG. 24, the catheter proximal end portion protrudes from the tunnel exit 126 and the excess catheter length E has been removed. Distal hub portion 162 and locking member 164 are slid onto the resultant catheter proximal end, and extension tube assembly 158 with proximal hub portion 160 is affixed to the catheter and the distal hub portion, as previously described with respect to FIG. 22. Locking member 164 is compressed by both the distal and proximal hub portions to grip the catheter proximal end within the completely assembled hub 152, assuredly securing the extension tube assembly to the catheter to complete the assembly.

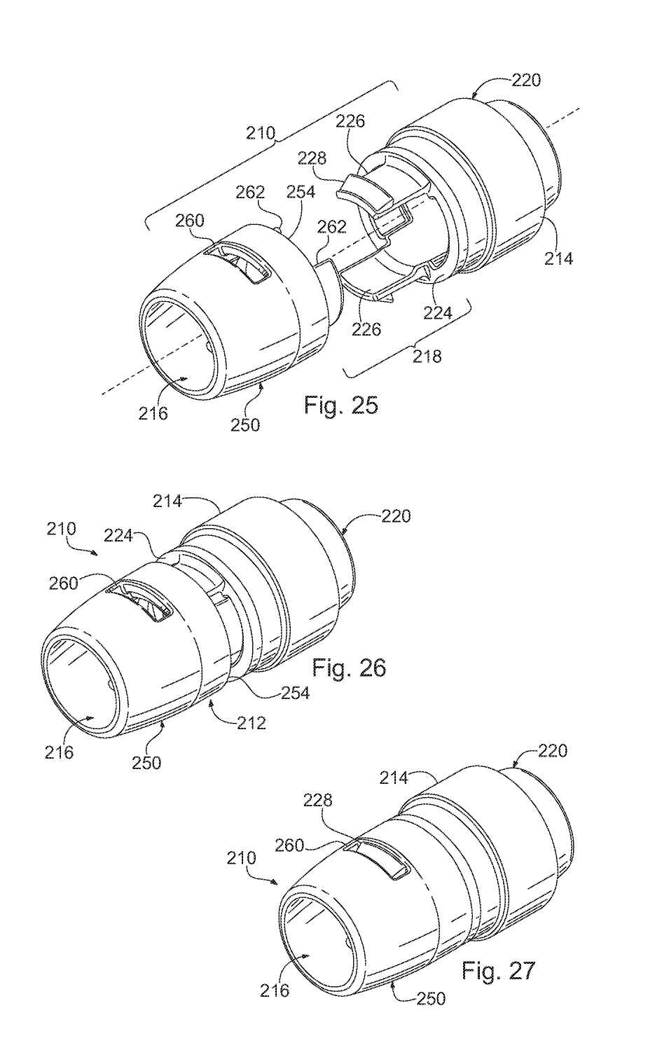

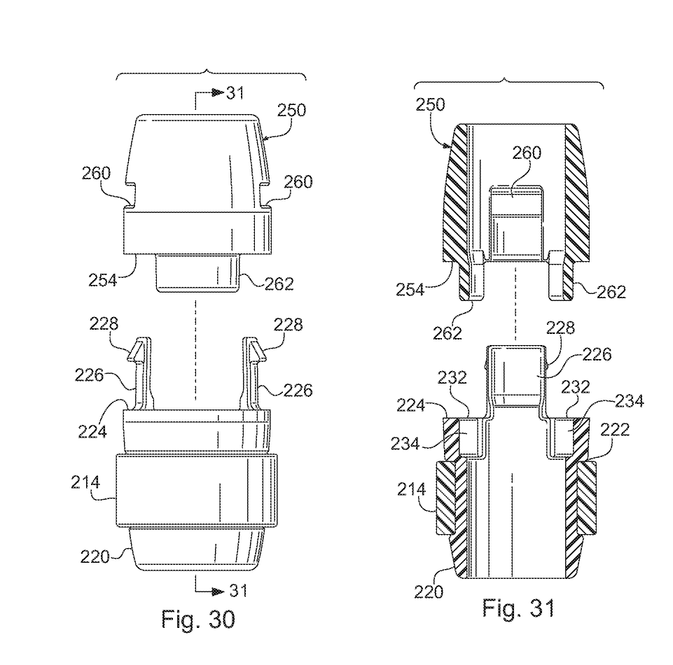

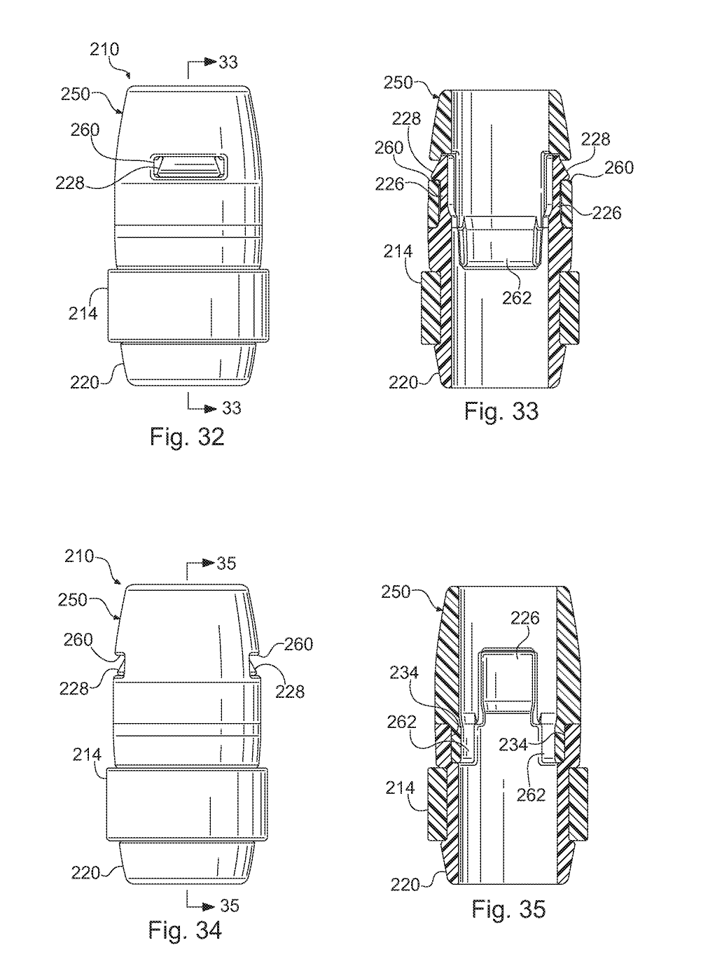

Referring to FIGS. 25-35, a cuff 210 in accordance with another exemplary embodiment of the invention is illustrated. Cuff 210 has a body section 212 and an outer covering 214 of tissue-ingrowth material bonded thereto. The cuff body section 212 may be of a biocompatible material such as polyethylene or polypropylene and defines a passageway 216.

FIGS. 25-27 illustrated the exemplary embodiment of cuff 210 wherein the cuff body section comprises a first portion 220 with a first mating interface 224 and a second portion 250 with a second mating interface 254. In the present embodiment, the outer covering 214 is provided about only one of the portions 220. As shown in FIG. 29, the covering 214 is received in and extends from an annular groove 222 formed in the outer surface of the first portion 220.

First and second portions 220, 250 are securable to each other at first and second interfaces 224, 254 by a latching arrangement 218. First portion 220 includes at least one, and preferably two opposed, latch arms 226 projecting axially from first interface 224 and having latching projections 228 at free ends thereof. Second portion 250 includes second interface 254 having a latch-receiving aperture 256 for each latch arm 226. Each aperture 256 communicates with a latch windows 260 into which the respective latching projection 228 of the latch arm 226 becomes latchable as will be explained hereinbelow.

Cuff 210 also includes a catheter-gripping arrangement defined adjacent first and second interfaces 224, 254. Second portion 250 includes at least one, and preferably two opposed, camming arms 262 projecting axially from second interface 254, while first portion 220 includes a camming arm-receiving groove 232 (see FIG. 31) for receipt of a respective camming arm 262. It is preferable that passageway 216 of the cuff be so dimensioned with respect to the catheter's outer diameter, that a slight amount of friction tends to inhibit slightly the ability of the cuff to slide freely along the catheter.

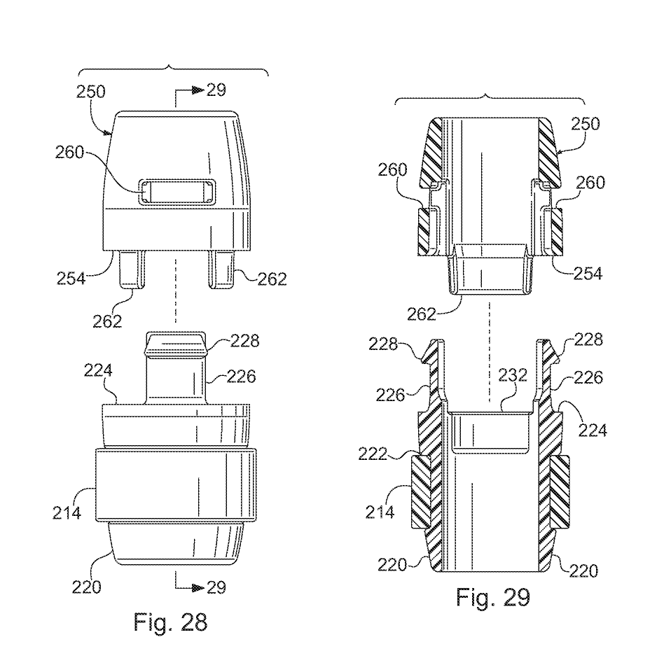

With reference now to FIGS. 28 to 31, the relationship of the latching and catheter-gripping structures of first and second portions 220, 250 are described in greater detail. In FIGS. 28 and 31, the camming arms 262 extend adjacent to passageway 126 and are spaced from one another an initial natural, non-urged distance D as shown in FIG. 31. The arms 262 are deflectable radially inwardly as the camming arms 262 are received into respective apertures 232 to a smaller distance D2, as shown in FIG. 35, which is smaller than the diameter of the catheter. The inside surface 234 of each aperture 232 is tapered inwardly to urge the respective camming arm 262 radially and are thereby urged against and into adjacent portions of catheter sidewall to establishing a gripping of the catheter by the cuff 210.

The cuffs of the present invention are placeable onto a catheter by the practitioner at a practitioner-selected location along the catheter, rather than be fixed into position on the catheter as conventional, giving the practitioner options with respect to locating the eventual subcutaneous tunnel. The cuffs of the present invention, coupled with recently developed hub and extension tube assemblies that may be affixed to the proximal end of the catheter by the practitioner after implantation and tunneling, enables the catheter to be manufactured to a deliberately overlong length with the expectation that the practitioner will trim the excess length once the practitioner is at the bedside of a particular patient. This in turn enables the use of one length of catheter to be used with practically any patient, rather than requiring an inventory of many different lengths of catheters in order to accommodate patients with particular attributes, conditions or situations.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A tissue-ingrowth cuff for an implantable catheter, comprising:
a body including first and second body portions, each body portion defining a through passage, the through passages defining a catheter-receiving passageway through the body when the first and second body portions are interconnected, with tissue-ingrowth material disposed on an outwardly facing surface of the body;
a locking arrangement actuatable to lock the first and second body portions relative to one another in a final locking position; and
a catheter-gripping arrangement comprising at least one catheter-gripping member defined by the first body portion and having a free engaging end and a complementary catheter-gripping actuator defined by the second body portion, the at least one catheter-griping member having a non-urged position wherein is free engaging end is radially clear of the catheter-receiving passageway,
wherein upon actuation of the locking arrangement to lock the first and second body portions relative to one another in a final locking position, each at least one catheter-gripping member is engaged by a respective complementary catheter-gripping actuator which urges the catheter-gripping member radially inwardly such that the at least one catheter-gripping member extends radially into the passageway.

2. The tissue-ingrowth cuff of claim 1 wherein the locking arrangement includes at least one locking section defined along a mating interface surface of one of the first and second body portions and associated with a complementary locking section defined along a mating interface surface of the other of the first and second body portions.

3. The tissue-ingrowth cuff of claim 2, wherein each at least one locking section is a latch arm projecting axially from the mating interface surface of one of the first and second body portions to a latching projection at its free end, and each at least one complementary locking section is an aperture defined axially into the mating interface surface of the other body portion, with at least one latching window in communication therewith associated with the latching projection of a respective latch arm to enable latching together the first and second body portions.

4. The tissue-ingrowth cuff of claim 3, wherein the locking arrangement includes a pair of latch arms and a pair of complementary latch apertures.

5. The tissue-ingrowth cuff of claim 3, wherein each of the at least one complementary locking section defines a pair of latching windows spaced apart axially, with one of the latching windows defining the final locking position and the other latching window defining an intermediate locking position.

6. The tissue-ingrowth cuff of claim 5, wherein, in the intermediate locking position, each at least one catheter-gripping member is urged radially inwardly into the passage an amount less than it is urged in the final locking position.

7. The tissue-ingrowth cuff of claim 1, wherein each body portion through passage is configured such that each body portion is slidable along the catheter prior to coupling together of the first and second body portions with the locking arrangement in the final locking position.

8. The tissue-ingrowth cuff of claim 1, wherein each at least one complementary catheter-gripping actuator is a projection extending from an interface surface of the second body portion and each at least one catheter-gripping member is an inner wall portion of an aperture in an interface surface of the first body portion.

9. The tissue-ingrowth cuff of claim 1, wherein each at least one catheter-gripping member is a projection extending from an interface surface of the first body portion and each at least one complementary catheter-gripping actuator is a wall surface of an aperture in an interface surface of the second body portion.

10. The tissue-ingrowth cuff of claim 1, wherein the first body portion is configured to be positioned on the catheter proximally of the second body portion.

11. The tissue-ingrowth cuff of claim 1, wherein the catheter-gripping arrangement includes two opposed catheter-gripping members and in the non-urged position, the opposed free engaging ends are spaced apart a first distance and when the locking arrangement is in the final locking position, the opposed free engaging ends are spaced apart a second distance which is less than the first distance.

12. The tissue-ingrowth cuff of claim 11, wherein the locking arrangement is configured to be affixed in an intermediate locking position and when when the locking arrangement is in the intermediate locking position, the opposed free engaging ends are spaced apart a third distance which is greater than the second distance and less than or equal to the first distance.

13. A method of implanting and anchoring a catheter in the vasculature of a patient, comprising the steps of:
extending a catheter through a passageway of a tissue-ingrowth cuff, the catheter having at least one lumen extending therethrough, a distal portion to be implanted within the vasculature of a patient, and a proximal portion to extend externally of the patient; and the cuff having a first condition wherein the cuff is slidable along the catheter and a second condition wherein the cuff self-clamps in position to and along the catheter;
sliding the cuff along the catheter to a first location approximately near a desired final location for the cuff;
selecting a location and length for a subcutaneous tunnel through which the catheter proximal portion will extend to anchor the catheter in position after implantation in the vasculature of the patient;
selecting a desired final location for the cuff corresponding to a position within the planned subcutaneous tunnel through which the catheter proximal portion will extend;
sliding the cuff along the catheter to the desired final location along the catheter;
activating the cuff to a condition wherein the cuff self-clamps to the catheter at said desired final location;
implanting the catheter distal portion into the vasculature of the patient; and
extending the catheter proximal portion through a subcutaneous tunnel such that the cuff is located within the subcutaneous tunnel.

14. The method of claim 13, wherein the cuff comprises two annular cuff portions which are slidable along the catheter prior to activating of the cuff, and the step of activating the cuff comprises urging the two cuff portions toward each other such that the cuff portions lock in a final locking position.

15. The method of claim 14, wherein the two annular cuff portions are connectable in a non-activating position such that the cuff portions are slidable along the catheter as a single unit.

16. The method of claim 13, further comprising the step of positioning a friction sleeve about the catheter proximate the desired final location prior to sliding the cuff to the desired final location.

17. The method of claim 13, wherein extending of the proximal end is done through a retrograde tunneling procedure.

18. The method of claim 13, wherein extending of the proximal end is done through a antegrade tunneling procedure.

19. A tissue-ingrowth cuff for an implantable catheter, comprising:
a body portion having an outer covering of tissue-ingrowth material, the body portion being annular and having a passageway therethrough so to be positionable around and slidable along a catheter to a desired position when in an unactuated condition, and the body portion being actuatable after positioning to achieve a catheter-gripping condition preventing relative movement of the catheter and the body portion.

20. A method of placing the tissue-ingrowth cuff of claim 19 onto an implantable catheter, comprising the steps of:
placing the cuff onto and around the catheter;
positioning the cuff at a selected position along the catheter by sliding the cuff therealong when the cuff is in an unactuated condition; and
actuating the cuff thereby securing the cuff in position along the catheter and preventing further movement of the catheter with respect to the cuff.
